(19) United States
(12) Patent Application Publication
(10) Pub. No.: US 2024/0312602 A1
(43) Pub. Date: Sep. 19, 2024

(54) COMPUTING SYSTEM FOR PROVIDING A MAPPING OF A PHYSICAL QUANTITY ON A BIOLOGICAL TISSUE AND METHOD THEREOF

(71) Applicants: Siemens Healthineers AG, Forchheim (DE); King's College London, London (GB)

(72) Inventors: Raphael Tomi-Tricot, ISSY-LES-MOULINEAUX (FR); Shaihan Malik, London (GB); Michael Dubiner, London (GB); Radhouene Neji, London (GB); Joseph V. Hajnal, London (GB)

(21) Appl. No.: 18/607,527

(22) Filed: Mar. 17, 2024

(30) Foreign Application Priority Data
Mar. 17, 2023 (EP) 23162592.2

(51) Int. Cl. G16H 30/00; G01R 33/44; G01R 33/48

(52) U.S. Cl. CPC G16H 30/00; G01R 33/443; G01R 33/48

(57) ABSTRACT

A computing system for providing a mapping of a physical quantity on a biological tissue includes a data interface configured to obtain data from the physical quantity on different spatial points of the biological tissue. The computing system includes a computation module having: a digitization unit configured to produce a digitized representation of the biological tissue in voxels and/or pixels; a concatenation unit configured to spatially correlate the voxels and/or pixels of the produced digitized representation of the biological tissue; and a regression unit configured to process the information of the spatially correlated voxels and/or pixels and generate a regression analysis of the physical quantity on the biological tissue. The computing system includes an output data interface configured to, based on the generated regression analysis, provide a mapping of the physical quantity on the biological tissue. A value of the physical quantity is assigned to each voxel and/or pixel.

This application claims the benefit of European Patent Application No. EP 23162592.2, filed on Mar. 17, 2023, which is hereby incorporated by reference in its entirety.

### BACKGROUND

The present embodiments relate to a computing system for providing mappings of physical quantities present on biological tissues. The present embodiments further relate to a computer-implemented method for providing such mappings.

The present embodiments are mostly described with respect to the determination of radiofrequency (RF) magnetic fields in the context of magnetic resonance imaging (MRI), especially in ultrahigh field MRI with parallel RF transmission, but the principles of the embodiments have a broader scope and apply equally well to other medical and/or clinical applications.

Herein and in the forthcoming, independent of the grammatical term usage, individuals with male, female, or other gender identities are included within the term.

A wide variety of advanced medical techniques are based on reliable determinations of the physical properties on biological tissues. Examples thereof are the determination of the temperature distribution of different organs (e.g., liver, brain, kidney) during a resection, the velocity field of corporal fluids (e.g., in the heart) or the determination of magnetic fields in soft tissue (e.g. during a magnetic resonance scan).

Quite generically, a determination of physical quantities on biological tissues involves measurement techniques that require long acquisition times. Magnetic resonance imaging (MRI) provides a number of examples of this sort. MRI is a non-invasive medical imaging technique for the examination of soft body tissues based on the detection of the relaxation times of proton spins, which originate as a response to pulses of computer-generated radio waves. Data acquisition may take on the order of minutes, during which patients are requested to do breath holds in order to reduce abdominal movement that makes it hard to image structures such as the heart or the blood, which have rather short characteristic times.

In order to ameliorate this problem, different measurement techniques have been developed, with the aim to reduce the acquisition time at the expense of accuracy, hoping to reach an acceptable balance between the two.

In MRI, such a situation takes place in the evaluation of the radio-frequency magnetic field B₁. One measurement mechanism is Actual Flip angle Imaging (AFI), which is very accurate but requires a lengthy acquisition time (e.g., of the order of minutes). Measurement alternatives are provided by different variants based on Fast Low Angle Shot magnetic resonance imaging (e.g., FLASH imaging). Such alternatives require acquisition times of the order of seconds, but in some cases (e.g., in MRI with ultrahigh magnetic fields), where the RF magnetic field is rather inhomogeneous over the biological tissue, the accuracy of conventional FLASH imaging methods turns out to be insufficient. In Sedlacik et al., “Calibration of saturation prepared turbo FLASH B₁⁺ maps by actual flip angle imaging at 7T,” Proceedings of ISMRM, 2022, p. 2867, an improved method was proposed, consisting in remapping the measured RF magnetic field B₁. Despite being an improvement, the resulting accuracy was spatially dependent, since the inaccuracies inherent in the turbo FLASH method are themselves spatially dependent, and this is not captured by the linear mapping used in Sedlacik et al.

Finding a compromise between acquisition time and accuracy is a generic problem that arises not only in the determination of magnetic fields in the context of MRI, but also in other medical techniques that deal with the determination of physical quantities on biological tissue.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a computing system for an accurate and time-efficient determination of a physical quantity on a biological tissue, such as soft biological tissues, is provided. The present embodiments are focused on an accurate and time-efficient determination of the radiofrequency magnetic field B₁ in the context of MRIs of mammal organs such as the brain.

A first aspect of the present embodiments provides a computing system for providing a mapping of a physical quantity on a biological tissue, including an input data interface configured to obtain data from the physical quantity on different spatial points of the biological tissue, and a computation module. The computation module includes at least: (a) a digitization unit configured to produce a digitized representation of the biological tissue in voxels and/or pixels, where, based on the obtained data, a subset of the voxels and/or pixels is assigned a value of the physical quantity; (b) a concatenation unit configured to spatially correlate the voxels and/or pixels of the produced digitized representation of the biological tissue; and (c) a regression unit configured to process the information of the spatially correlated voxels and/or pixels and generate a regression analysis of the physical quantity on the biological tissue. The computer system further includes an output data interface configured to, based on the generated regression analysis, provide a mapping of the physical quantity on the biological tissue. A value of the physical quantity is assigned to each voxel and/or pixel.

Biological tissue refers in the present embodiments mostly to soft biological tissue, such as organs such as, for example, the brain, the kidney, the heart, or the liver of mammals and humans.

A physical quantity in the context of the present embodiments is to be understood as any physical property that may be quantified by a measurement and expressed as a scalar field (e.g., temperature field) or a vector field (e.g., magnetic field or velocity field).

Herein and in the forthcoming, a mapping is a correspondence between a physical quantity and the spatial points of a biological tissue, such that there is a value of the physical quantity associated with each spatial point. The mapping provided by the present embodiments may also be referred to as a remapping, a correction, or a calibration, since the mapping corrects or calibrates the data from the physical quantity obtained via the input data interface.

A digitization of the physical tissue is a mathematical representation or discretization of the physical tissue in terms of pixels (e.g., if the digitization is in two dimensions) or in voxels (e.g., if the digitization is in three dimensions).

A spatial correlation of the voxels and pixels includes any procedure or mechanism by which the voxels and pixels are concatenated in a position space with respect to a system of reference, such that one may define, for example, relative distances between the different voxels and/or pixels.

A regression analysis refers to any mathematical procedure or algorithm defining an objective function involving the physical quantity at the different spatial points of the biological tissue that is to be extremized (e.g., maximized or minimized).

The computation module and the different units mentioned in this application are broadly understood as entities capable of acquiring, obtaining, receiving, or retrieving generic data and/or instructions through a user interface and/or programming code and/or executable programs or any combination thereof. The computation module and the different units are configured to run programming code and executable programs and to deliver the results for further processing.

The computation module and units included within the computation module, or parts thereof, may therefore each contain, at least, a central processing unit, CPU, and/or at least one graphics processing unit, GPU, and/or at least one field-programmable gate array, FPGA, and/or at least one application-specific integrated circuit, ASIC and/or any combination of the foregoing. Each may further include a working memory operatively connected to the at least one CPU and/or a non-transitory memory operatively connected to the at least one CPU and/or the working memory. Each may be implemented partially and/or completely in a local apparatus and/or partially and/or completely in a remote system such as by a cloud computing platform.

All of the elements of the computing system may be realized in hardware and/or software, cable-bound and/or wireless, and in any combination thereof. Any of the elements may include an interface to an intranet or the Internet, to a cloud computing service, to a remote server, and/or the like.

For example, the computing system of the invention may be implemented partially and/or completely in a local apparatus (e.g., a computer) in a system of computers and/or partially and/or completely in a remote system such as a cloud computing platform.

In systems based on cloud computing technology, a large number of devices are connected to a cloud computing system via the Internet. The devices may be located in a remote facility connected to the cloud computing system. For example, the devices may include, or consist of, equipment, sensors, actuators, robots, and/or machinery in an industrial set-up(s). The devices may be medical devices and equipment in a healthcare unit. The devices may be home appliances or office appliances in a residential/commercial establishment.

The cloud computing system may enable remote configuring, monitoring, controlling, and maintaining connected devices (also commonly known as ‘assets’). Also, the cloud computing system may facilitate storing large amounts of data periodically gathered from the devices, analyzing the large amounts of data, and providing insights (e.g., Key Performance Indicators, Outliers) and alerts to operators, field engineers, or owners of the devices via a graphical user interface (e.g., of web applications). The insights and alerts may enable controlling and maintaining the devices, leading to efficient and fail-safe operation of the devices. The cloud computing system may also enable modifying parameters associated with the devices and issues control commands via the graphical user interface based on the insights and alerts.

The cloud computing system may include a plurality of servers or processors (also known as ‘cloud infrastructure’) that are geographically distributed and connected to each other via a network. A dedicated platform (hereinafter referred to as “cloud computing platform”) is installed on the servers/processors for providing above functionality as a service (hereinafter referred to as “cloud service”). The cloud computing platform may include a plurality of software programs executed on one or more servers or processors of the cloud computing system to enable delivery of the requested service to the devices and its users.

One or more application programming interfaces (APIs) are deployed in the cloud computing system to deliver various cloud services to the users.

A second aspect of the present embodiments provides a computer-implemented method for providing a mapping of a physical quantity on a biological tissue, including the following acts: (a) obtaining data from the physical quantity on different spatial points of the biological tissue; (b) producing (S2) a digitized representation of the biological tissue in voxels and/or pixels, where a subset of the voxels and/or pixels gets assigned a value of the physical quantity based on the acquired data; (c) spatially correlating the voxels and/or pixels of the produced digitized representation of the biological tissue; (d) generating, based on the information of the spatially correlated voxels and/or pixels, a regression analysis of the physical quantity on the biological tissue; and (e) providing, based on the generated regression analysis, a mapping of the physical quantity on the biological tissue, where a value of the physical quantity is assigned to each voxel and/or pixel.

For example, the method according to the second aspect of the present embodiments may be carried out by the system according to the first aspect of the present embodiments. The features and advantages disclosed herein in connection with the computing device are therefore also disclosed for the method, and vice versa.

According to a third aspect, the present embodiments provide a computer program product including executable program code configured to, when executed, perform the method according to the second aspect of the present embodiments.

According to a fourth aspect, the present embodiments provide a non-transient computer-readable data storage medium including executable program code configured to, when executed, perform the method according to the second aspect of the present embodiments.

The non-transient computer-readable data storage medium may include, or consist of, any type of computer memory (e.g., semiconductor memory such as a solid-state memory). The data storage medium may also include, or consist of, a CD, a DVD, a Blu-Ray-Disc, an USB memory stick, or the like.

According to a fifth aspect, the present embodiments provide a data stream including, or configured to generate, executable program code configured to, when executed, perform the method according to the second aspect of the present embodiments.

According to a sixth aspect, the present embodiments provide a medical scanning system including a magnetic resonance image scanner. The magnetic resonance image scanner is configured to perform magnetic resonance imaging scans (e.g., ultrahigh field magnetic resonance imaging scans with parallel radio-frequency imaging). The medical scanning system also includes the computing system, which is configured to obtain data corresponding to the radio-frequency magnetic field from the magnetic resonance image scanner.

The computing system may be implemented (e.g., as part of or connected to) as an MRI scanner. In this way, the data of the RF magnetic field obtained, for example, using FLASH imaging may be quickly processed, and images of the mapping (also referred to as remapping, correction, or calibration) may be visualized in, for example, a video display unit. The computing system of the present embodiments may also be connected to computing devices responsible to produce the MRI images, feeding the computing devices with the mappings of the B₁ magnetic field. Parts of the computing system of the present embodiments may also be implemented in a cloud computing platform.

One of the main ideas underlying the present embodiments is to provide a computing system that is configured to generate, based on data coming from the evaluation of a physical quantity (e.g., temperature, magnetization) on a biological tissue, a more accurate mapping (or prediction of the spatial distribution) of the physical quantity based on the results of a regression analysis. The regression analysis uses as input the spatial information and correlations between the different points of a digitized representation of the biological tissue. In the particular example of ultrahigh field MRI, the obtained data may be generated using, for example, saturation-prepared turbo FLASH (satTFL) imaging, from which a more accurate mapping of the B₁ magnetic field (e.g., a mapping closer to the actual B₁ magnetic field) may be generated with a regression analysis. This regression analysis, thanks to the implemented information on spatial correlations, is especially adapted to map fields with spatial inhomogeneity. This correction or calibration of the satTFL data may be generated in a matter of seconds.

The computing system of the present embodiments includes a computation module that is configured to: (a) provide a digital representation of the biological tissue, where at least some of the pixels (or voxels) are assigned a value of the physical quantity; (b) spatially correlate the pixels (or voxels); and (c) generate a regression analysis from which a mapping is provided, where all the pixels (or voxels) are assigned a value of the physical quantity.

The device as described above allows for a simple implementation of a computer-implemented method including a number of acts. In one act, data from the physical quantity on different spatial points of a biological tissue is obtained. This data may be generated by any process that may measure the physical quantity. In another act, the biological tissue is digitized, where a subset of the ensuing voxels or pixels gets assigned a value of the physical quantity. In a subsequent act, spatial information of the voxels or pixels of the digitized biological tissue is provided, such that the values of the physical quantity are spatially correlated. This information is then used in another act to generate a regression. In a subsequent act, the regression is used to provide a mapping that associates every point of the biological tissue with a corresponding value of the physical quantity.

One advantage of the present embodiments is that the provided mapping furnishes a determination of a physical quantity on a biological tissue that is accurate and may be performed in a time-efficient fashion. As an example, in the context of MRI scans, the determination of the RF magnetic field B₁ may be performed accurately with Actual Flip angle Imaging (AFI), but the time required spans over minutes. More time-efficient solutions are provided by Fast Low Angle Shot magnetic resonance imaging (FLASH imaging) (e.g., with satTFL) at the expense of a lower accuracy. The present embodiments may be provided with the fast-generated data from satTFL as input and produce a more accurate mapping (e.g., a calibration or correction of the satTFL data that falls closer to the actual spatial distribution of the B₁ field) in a matter of seconds, which makes the present embodiments amenable for its use in the clinical workflow.

A further advantage of the present embodiments is that, especially due to the implementation of a spatial correlation between the voxels or pixels of the digitized version of the biological tissue, the regression provided by the system of the present embodiments is very robust when applied to the prediction of physical quantities with spatial-dependent profiles. Accordingly, when the present embodiments are used in these cases, spatial-dependent inaccuracies may be efficiently reduced. Such a situation arises in ultrahigh field MRI, where the RF magnetic field B₁ is known to be spatially inhomogeneous.

According to some embodiments, refinements, or variants of embodiments, the computation module of the present embodiments further includes a performance assessment unit that is configured to evaluate the accuracy of the provided mapping based on a ground truth.

The performance assessment unit may be configured to assess the system performance quantitatively by using different figures of merit or metrics. For example, the R² score, defined as

$${R}^{2} = 1 - \frac{{\sum }_{i} {( {κ}_{i} - {\hat{κ}}_{i} )}^{2}}{{\sum }_{i} {( {κ}_{i} - \overline{κ} )}^{2}}$$

may be determined, where \hat{κ}ᵢ is the value of the physical quantity under consideration at voxel or pixel i coming from the generated mapping; κᵢ is the value of the physical quantity under consideration at voxel or pixel i from the ground truth; and κ̅ is the value of the physical quantity under consideration from the ground truth averaged over all voxels or pixels.

Alternatively, the normalized root mean-squared error (NRMSE) may be used, defined as

$$N RMS E = \sqrt{\frac{{\sum }_{i} {( {κ}_{i} - {\hat{κ}}_{i} )}^{2}}{{\sum }_{i} {( {κ}_{i} )}^{2}}}$$

The performance assessment unit may be used to analyze the results of each regression and further improve the involved algorithms.

According to some embodiments, refinements, or variants of embodiments, the physical quantity is the radiofrequency magnetic field generated during magnetic resonance imaging.

As already mentioned in the foregoing, a particularly suited application of the present embodiments arises in the context of magnetic resonance imaging (MRI), and, for example, in the mapping of the radiofrequency (RF) magnetic field B₁ (also referred to as B₁⁺). In response to this magnetic field, the spins of the protons in the biological tissue change their alignment and subsequently generate a signal via induction, allowing imaging. The amount of change of the alignment depends on B₁; hence, there is often a need to map this field for calibration or correction purposes.

According to some embodiments, refinements, or variants of embodiments, the magnetic resonance imaging includes ultrahigh field magnetic resonance imaging with parallel transmission imaging radiofrequency.

Ultrahigh field MRI denotes MRI with static magnetic fields at or above 7 Tesla, requiring RF magnetic fields B₁ with frequencies at or above 298 MHz. At these frequencies, the spatial inhomogeneities of the B₁ along the examined biological tissue are known to be substantial and are to be considered. Parallel transmission or multichannel transmission imaging RF is a technique that is used to accelerate the acquisition of MRI data. This is achieved by an arrangement of coils, each acting as a RF-transmitter and/or a RF-receiver. Together, the arrangement of coils builds a multichannel Tx/Rx array. The B₁ field experienced by the biological tissue is the superposition of such transmitted fields. The coils may be arranged so that the properties of the transmitted B₁ magnetic field are optimized (e.g., by reducing the inhomogeneities of the B₁ field on the biological tissue).

According to some embodiments, refinements, or variants of embodiments, the obtained data of the radiofrequency magnetic field is generated following a Fast Low Angle Shot (FLASH) magnetic resonance imaging.

Saturation-prepared Turbo-FLASH magnetic resonance imaging (or satTFL) is a technique to determine the B₁ transmission field that combines a high flip angle saturation pulse, followed by a train of low flip angle RF excitations with a low repetition time. The technique allows for a fast determination of the B₁ magnetic field (e.g., the acquisition time lasts a few seconds) with a satisfactory accuracy, and is already implemented in some scanning machines as part of the pre-scan adjustments. It is therefore a technique that fulfills the requirements of the present embodiments as a source of the data obtained by the input data interface. FLASH imaging is also known by some manufacturers as spoiled gradient echo (SPGR) or contrast-enhanced fast field echo (CE-FFE-T1 or T1-FFE).

According to some embodiments, refinements, or variants of embodiments, the ground truth includes a mapping of the radiofrequency magnetic field acquired with Actual Flip angle Imaging (AFI).

Actual Flip angle Imaging (AFI) may be currently considered a “gold standard”, in terms of accuracy, in the determination of the transmission RF magnetic field B₁, and AFI is therefore an optimal method to use as ground truth for the figures of merit used by the performance assessment unit to evaluate the accuracy of the mapping provided by the system of the present embodiments.

The AFI is very accurate but also has slow acquisition times. In order for the performance assessment unit to use AFI data, AFI from a database of recorded past MRIs may be used.

According to some embodiments, refinements, or variants of embodiments, the computation module further includes a feature generating unit, configured to compute the volume of the biological tissue and/or the spatial coordinates of its center of mass.

The spatial correlation of the voxels and pixels includes at least the identification of their three-dimensional Cartesian coordinates referred to a spatial system of reference. This local data may be complemented with global data describing the geometry of the biological tissue as a whole. In this respect, two possible parameters are the volume of the biological tissue and its center of mass.

According to some embodiments, refinements, or variants of embodiments, the regression unit further includes an artificial intelligence entity that is configured to implement at least one machine learning regression algorithm (e.g., a random forest tree algorithm and/or a gradient boosting algorithm).

Whenever herein an artificial intelligence entity is mentioned, it shall be understood as a computerized entity able to implement different data analysis methods broadly described under the terms artificial intelligence, machine learning, deep learning, or computer learning. The artificial intelligence entity may include any realization thereof able to perform a regression. Random forest tree (RFT) and gradient boosting (GB) are two examples of well-tested algorithms that are easy to implement, but the invention is not limited to RFT and GB, and, for example, other options (e.g., options including neural networks) are also possible.

According to some embodiments, refinements, or variants of embodiments, the machine learning regression algorithm uses as input a feature matrix including the spatial components of each voxel and/or pixel and the value of the physical quantity assigned to the respective voxel and/or pixel.

In examples of implementations of machine learning algorithms, the voxels and/or pixels are provided with a mask or an annotation, consisting of a feature matrix that combines local information corresponding to each voxel (or pixel) (e.g., geometrical information including the three-dimensional coordinates and an assigned value of the physical quantity). This local information may be combined with additional information (e.g., global information), such as the volume and center of mass of the biological tissue. These last two quantities may be provided by the feature generating unit.

According to some embodiments, refinements, or variants of embodiments, the computing system further includes an optimization unit that is configured to use the evaluation of the performance assessment unit to optimize the machine learning regression algorithm.

In these embodiments, data for the optimization may include stored data from previous MRIs or data from the artificial intelligence unit of systems connected to other scanning MRI machines. The optimization unit may be configured to implement different deep learning or reinforcement learning algorithms.

According to some embodiments, refinements, or variants of embodiments, the biological tissue is brain tissue. Until recently, ultrahigh field MRI applications were mostly devoted to brain scans, and the brain remains the organ where ultrahigh field MRI is best understood. In this context, the present embodiments may be used to help diagnose, for example, aneurysms of cerebral vessels, the occurrence of strokes, brain tumors, brain injury after trauma, multiple sclerosis, disorders of the eye, Alzheimer's disease, or epilepsy. Applications using functional MRI (fMRI) are also under the scope of the present embodiments.

According to some embodiments, refinements, or variants of embodiments, the computer-implemented method of the second aspect of the present embodiments further include: creating a feature matrix including the spatial components of each voxel and/or pixel and the value of the physical quantity assigned to it, where the feature matrix is used as input for generating the regression analysis.

In some embodiments, the feature matrix may also contain information about the volume of the biological tissue and/or the spatial coordinates of its center of mass. In the particular case of the mapping of the radiofrequency B₁ magnetic field in the context of ultrahigh field MRI, the use of a feature matrix with a combination of local information (e.g., voxel positions and their B₁ values) with global information (e.g., the volume of the brain and the position of its center of mass) turns out to be advantageous for improving the performance of some regression algorithms.

Although here, in the foregoing and also in the following, some functions are described as being performed by modules or units, it shall be understood that this does not necessarily mean that such modules or units are provided as entities separate from one another. In cases where one or more modules or units are provided as software, the modules or units may be implemented by program code sections or program code snippets, which may be distinct from one another but may also be interwoven or integrated into one another.

Similarly, in cases where one or more modules or units are provided as hardware, the functions of one or more modules or units may be provided by one and the same hardware component, or the functions of several modules or units may be distributed over a number of hardware components that need not necessarily correspond to the modules or units. Thus, any apparatus, system, method, and so on which exhibits all of the features and functions ascribed to a specific module or unit shall be understood to include, or implement, the module or the unit. For example, it is a possibility that all modules or units are implemented by program code executed by the computing device (e.g., a server or a cloud computing platform).

The above embodiments and implementations may be combined with each other as desired, as far as this is reasonable.

Further scope of the applicability of the present method and system will become apparent from the following figures, detailed description and claims. However, it should be understood that the detailed description and specific examples, while indicating embodiments of the invention, are given by way of illustration only, since various changes and modifications within the spirit and scope of the invention will become apparent to those skilled in the art.

### BRIEF DESCRIPTION OF THE DRAWINGS

Aspects of the present disclosure will be better understood with reference to the following figures.

FIG. 1 is a schematic depiction of a computing system for providing a mapping of a physical quantity on a biological tissue according to an embodiment;

FIG. 2 is a block diagram showing an example embodiment of a computer-implemented method for providing a mapping of a physical quantity on a biological tissue;

FIG. 3 is a schematic illustration of a medical scanning system including the computing system according to an embodiment, and a magnetic resonance imaging scanner;

FIG. 4 shows two plots, where the performance of two regression algorithms generated with a computing system according to an embodiment is compared to the performance of a conventional algorithm;

FIG. 5 shows two scattering plots, where the performance of regression algorithm according to an embodiment is compared with the performance of a conventional algorithm;

FIG. 6 shows two-dimensional projections of a brain B₁ map acquired via MRI using an embodiment of the computing system, compared with the ground truth and a state-of-the-art fitting technique;

FIG. 7 is a schematic block diagram illustrating a computer program product according to an embodiment of the third aspect of the present embodiments; and

FIG. 8 is a schematic block diagram illustrating a non-transitory computer-readable data storage medium according to an embodiment of the fourth aspect of the present embodiments.

### DETAILED DESCRIPTION

Parts in the different figures that correspond to the same elements have been indicated with the same reference numerals.

The components in the drawings are not necessarily to scale, emphasis being placed instead upon clearly illustrating the principles of the present disclosure. Likewise, certain components may be shown in generalized or schematic form in the interest of clarity and conciseness. In some instances, well-known structures and devices are shown in block diagram form in order to avoid obscuring the concepts of the present embodiments.

The numeration of the acts in the methods are meant to ease their description. The numeration does not necessarily imply a certain ordering of the acts. For example, a number of acts may be performed concurrently.

The detailed description includes specific details for the purpose of providing a thorough understanding of the present invention. However, it will be apparent to those skilled in the art that the present invention may be practiced without these specific details.

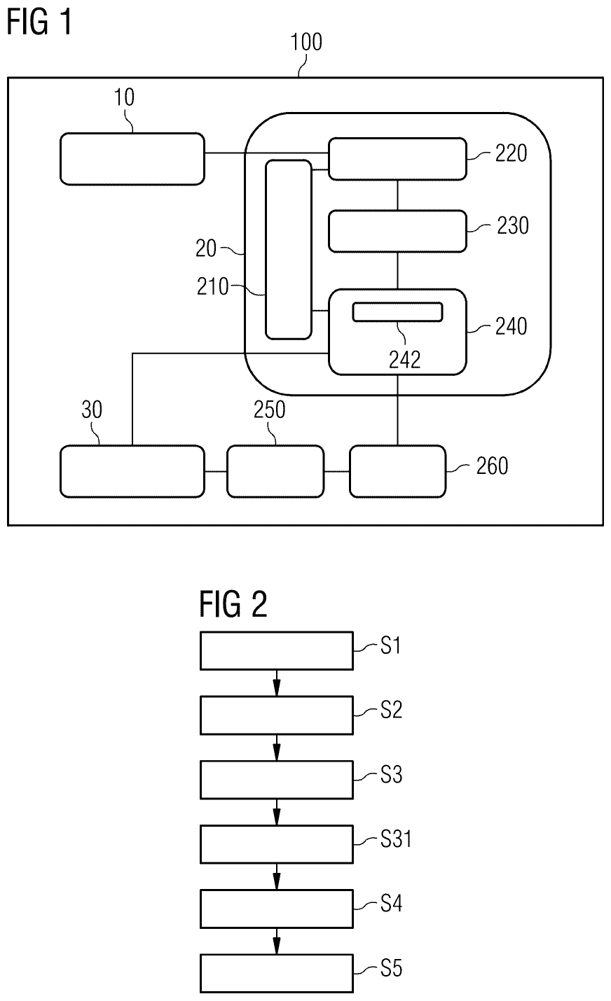

FIG. 1 shows a schematic depiction of a computing system 100 for providing a mapping of a physical quantity on a biological tissue according to an embodiment (e.g., the mapping of the radio-frequency magnetic field B₁ during an ultrahigh magnetic resonance imaging with parallel imaging of a brain). In the following, we will describe this particular implementation of the computing system of the present embodiments.

The computing system 100 depicted in FIG. 1 includes an input data interface 10, a computation module 20, an output data interface 30, a performance assessment unit 250, and an optimization unit 260.

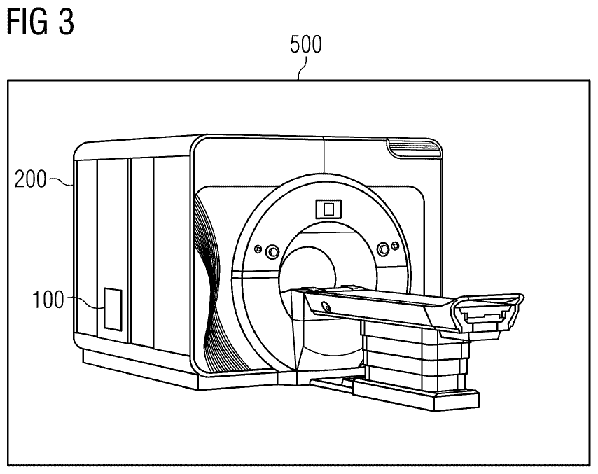

The input data interface 10 is configured to obtain data from the radio-frequency magnetic field B₁ on different spatial points of the brain. This data may originate, for example, from the magnetic resonance imaging scanner 200 depicted in FIG. 3, which may be endowed with a coil arrangement for parallel imaging (not shown in the figure) and a B₁ acquisition unit (not shown in the figure). The B₁ acquisition unit is configured to measure the B₁ magnetic field (e.g., with a Fast Low Angle Shot (FLASH) magnetic resonance imaging, such as a satTFL). The input data interface 10 may be connected to the B₁ acquisition unit of the magnetic resonance imaging scanner 200, wired or wireless or with a combination thereof.

The computation module 20 includes a feature generating unit 210, a digitization unit 220, a concatenation unit 230, and a regression unit 240.

The digitization unit 220 is configured to generate a digitized representation of the brain in voxels, where, based on the obtained data, a subset of the voxels and/or pixels are assigned a value of B₁ magnetic field. The assigned B₁ value is based on the data obtained through the input data interface 10.

The concatenation unit 230 is configured to spatially correlate the voxels of the generated digitized representation of the brain. This may be achieved by computing the three-dimensional coordinates rᵢ=(xᵢ, yᵢ, z₁) of each voxel i of the digitized representation of the brain with respect to a common system of reference, such that relative distances between the different voxels may be computed.

The regression unit 240 is configured to process the information of the spatially correlated voxels of the digitized representation of the brain and generate a regression analysis of the radio-frequency magnetic field B₁ on the brain.

In the implementation shown in FIG. 1, the regression unit 240 is further configured to implement an artificial intelligence entity 242 that is configured to implement at least one machine learning regression algorithm (e.g., a random forest tree algorithm and/or a gradient boosting algorithm).

The regression algorithms may use as input a feature matrix including the spatial components rᵢ of each voxel and/or pixel i and the value of B₁ assigned to the respective voxel and/or pixel i, together with global quantities such as the volume of the biological tissue and/or the spatial coordinates of its center of mass. The spatial components of the voxels and the corresponding values of the B₁ magnetic field give local information about the brain, which may be combined with global or topological information, such as the brain volume or the brain center of mass. In some embodiments, the computing system 100 includes a feature generating unit 210 that is configured to compute global quantities such as the brain volume and/or the spatial coordinates of its center of mass. In these embodiments, the feature generating unit 210 uses as input the information from the digitization unit 220. The global quantities computed by the feature generating unit 210 are then fed into the feature matrix to be used by the regression unit 240 as input.

The output data interface 30 is configured to provide a mapping of the B₁ magnetic field on the brain based on the regression analysis generated by the regression unit 240. Through the mapping, a value of the B₁ magnetic field is assigned to each voxel.

The embodiment illustrated in FIG. 1 further includes a performance assessment unit 250 that is configured to evaluate the accuracy of the mapping provided by the output data interface 30. This may be done by using different figures of merit, such as a R² score metric or a normalized root mean-square error (NRMSE), which compare the B₁ field on each voxel obtained from the mapping with a ground truth, such as the B₁ determination coming an Actual Flip angle Imaging (AFI).

Some embodiments, such as the one depicted in FIG. 1, also include an optimization unit 260 that is configured to use the evaluation of the performance assessment unit 250 to optimize the machine learning regression algorithm implemented by the regression unit 240.

FIG. 2 is a block diagram showing an example embodiment of a computer-implemented method for providing a mapping of a physical quantity on a biological tissue. The method may be implemented with the computing system 100 described with respect to FIG. 1. In the following, and for concreteness, the method will be applied to the determination of a mapping for the RF magnetic field B₁ during an MRI scan. The method includes a number of acts.

In one act S1, data from the RF magnetic field B₁ is obtained on different spatial points of the brain of a patient during an MRI pre-scan. The data may be produced using FLASH imaging, which has short acquisition times and good accuracy.

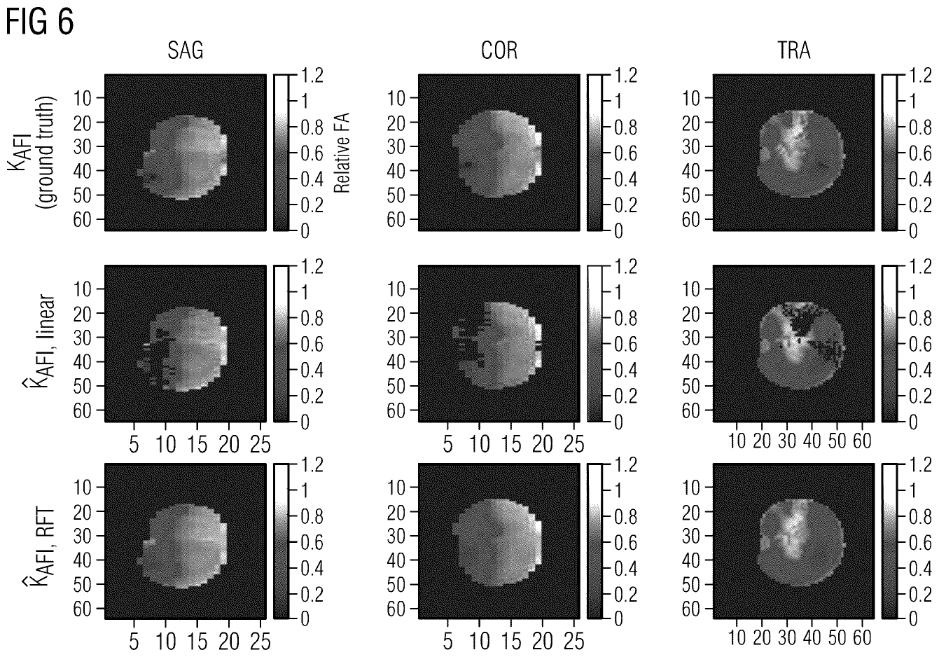

In another act S2, the brain is digitized, where at least a subset of the ensuing voxels gets assigned a value of the B₁ field. The subset of voxels that get an assigned value, and the specific assigned value, depend on the characteristics of the data obtained from the FLASH imaging. In some cases, not all the voxels get a value; in some other cases, the values of the B₁ magnetic fields are interpreted as null because of signal tolerances. In FIG. 6, some of these cases are discussed with respect to illustrative experiments.

In a subsequent act S3, the voxels of the digitized brain are spatially correlated. This may be done in some cases by assigning spatial coordinates rᵢ=(xᵢ,yᵢ,zᵢ) to each of the voxels i, with respect to a common spatial frame of reference.

The embodiment depicted in FIG. 2 shows act S31, in which a feature matrix is created. This feature matrix includes the spatial coordinates rᵢ=(xᵢ,yᵢ,zᵢ) of each voxel and the value of the B₁ magnetic field assigned to respective voxel. In some embodiments, the feature matrix may also contain additional information (e.g., the volume of the brain and/or the spatial coordinates of its center of mass).

This information is then used, in another act S4, to generate a regression analysis. This regression analysis may be implemented with machine learning regression algorithms. In some embodiments, these algorithms include a Random Forest Tree (RFT) algorithm and/or a gradient boosting (GB) algorithm. In some other embodiments, the machine learning algorithms may use as input a feature matrix including the spatial coordinates rᵢ of each voxel i, the value of the magnetic field B₁ assigned to each voxel i, the volume of the brain, and the spatial coordinates of the center of mass of the brain.

In a subsequent act S5, the generated regression analysis is used to provide a B₁ mapping that associates every point of the brain with a corresponding value of the B₁ magnetic field. As a result of the regression, even for voxels where B₁ values from the FLASH imaging are unavailable, the mapping may still make a prediction for those voxels using knowledge of their spatial location. The result of the regression is not only a smooth mapping, but one that, with the help of the deployed machine learning algorithms, is closer to the ground truth (e.g., more accurate) than the FLASH imaging and may be provided in a matter of seconds.

FIG. 3 is a schematic illustration of a medical scanning system 500 including the computing system according to an embodiment, and a magnetic resonance imaging scanner 200.

The magnetic resonance imaging scanner 200 may be a magnetic resonance scanner (e.g., configured to perform ultrahigh field magnetic resonance imaging scans with parallel radio-frequency imaging). An example thereof is the MAGNETOM Terra from Siemens Healthcare in Erlangen, Germany.

The computing system 100 is configured to obtain data corresponding to the radio-frequency magnetic field B₁ from the magnetic resonance imaging scanner 200. This data may be obtained with a Fast Low Angle Shot (FLASH) magnetic resonance imaging, such as satTFL.

In the embodiment illustrated in FIG. 3, the computing system 100 or parts thereof are integrated in the magnetic resonance imaging scanner 200. Parts of the computing system 100 that are not integrated in the magnetic resonance imaging scanner 200 may be connected to the magnetic resonance imaging scanner 200.

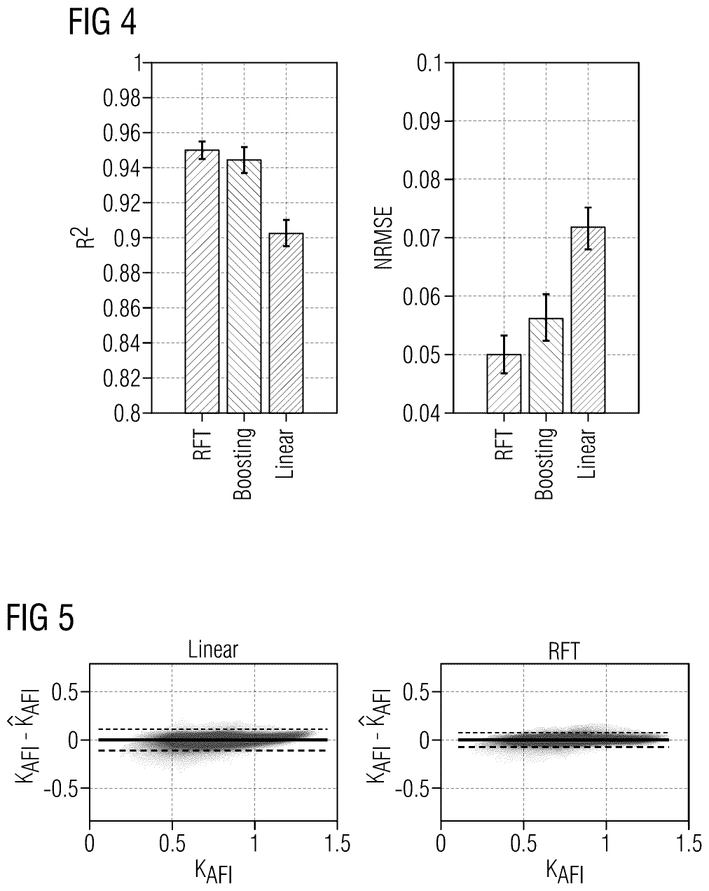

FIG. 4 shows two plots, where the performance of two regression algorithms generated with a computing system according to an embodiment is compared to the performance of a conventional algorithm.

The plots of FIG. 4 correspond to a test performed with 26 healthy volunteers, who underwent a scan on the MAGNETOM Terra 7T scanner of Siemens Healthcare in Erlangen, Germany. The parallel imaging was arranged with a 8Tx/32Rx head coil with 8 transmission channels and 32 receiving channels.

Sets of brain B₁ maps were acquired for each volunteer using Actual Flip angle Imaging (AFI) and saturation-prepared turbo FLASH (satTFL) imaging, with channels combined in circular polarization with matched imaging positions, fields of view (FOV), and resolutions. The AFI consisted of a FOV of 256×256×240 mm³, a 64×64×48 voxel matrix, nominal flip angle of 60°, and a typical scanning time (per channel) of 3 min 16 s. In turn, the satTFL comprised 25 slices of 5 mm thickness with a 5 mm spacing, in-plane FOV of 256×256 mm², a 64×64×25 voxel matrix, a nominal flip angle of 90°, and a typical scanning time (per channel) of 8 s.

Each of the scanned brains were digitized. A feature matrix was used for each scan, including the spatial positions of each voxel, the magnitude value of the B₁ field on each voxel, the volume of the brain, and the position of its center of mass. This 8-dimensional feature matrix was fed into a Random Forest Tree (RFT) algorithm and a Gradient Boosting (GB) algorithm. The algorithms were trained using the AFI results as ground truth. The training consisted of 26 folds, where for each fold, one of the 26 datasets was excluded from the training and left as a test dataset. The left-out dataset was predicted after each training fold and compared to the ground truth (the AFI value). The algorithm performance was assessed quantitatively using as figures of merit an R² and a NRMSE metric.

FIG. 4 shows the results obtained for the R² (left panel) and the NRMSE (right panel) metrics for the RFT and the GB algorithms, averaged over the 26 folds.

The RFT algorithm was trained with a training configuration of 362 trees, 242460 maximum tree branch splits, a minimum of 5 samples per leaf node, and 3 features sampled at each tree split. The GB algorithm was implemented with a least squares loss function and trained with a training configuration of 362 trees, 242460 maximum tree branch splits, a minimum of 5 samples per leaf node, and a learning rate of 0.1.

In FIG. 4, the results obtained for the RFT and GB algorithms are compared with the linear fitting method of Sedlacik et al., “Calibration of saturation prepared turbo FLASH B1+ maps by actual flip angle imaging at 7T,” Proceedings of ISMRM, 2022, p. 2867, noted as “linear” in the plots. Sedlacik et al. use only the values of the B₁ magnetic field as feature matrix, while the computing system of the present embodiments also takes into account geometric features, both local (e.g., the spatial coordinates of the voxels) and global (e.g., the volume of the brain and/or the coordinates of its center of mass), and concatenates the geometric features.

The plots in FIG. 4 show a superior performance of the system of the present embodiments with respect to the prior state of the art, with a slightly better performance obtained with RFT.

An overfitting study of the regression algorithms was also performed by comparing the R² score shown in FIG. 4 with the values obtained when test datasets to be predicted belonged to the training set. The average R² for RFT moved from 0.951 to 0.977, while for GB, the average R² moved from 0.945 to 0.998. The smaller increase for RFT indicates that RFT is more robust against overfitting.

The algorithms of the computing system of the invention performed with an average accuracy of 0.95 when compared with the AFI data (e.g., the ground truth) and provided the mapping (e.g., the correction or calibration of the satTFL data) for each volunteer in a matter of 2 seconds, which is quick enough to be used in clinical workflows.

FIG. 5 shows two scattering plots, where the performance of regression algorithm according to an embodiment is compared with the performance of a conventional algorithm. The results in FIG. 5 correspond to the same case study already detailed with respect to FIG. 4. The (scattered) plots of FIG. 5 show the difference between \hat{κ}_{AFI} and \hat{κ}_{AFI} (e.g., the difference between the B₁ magnetic field and the ground truth (obtained with AFI) for each of the voxels of all the tested brains) normalized to the nominal flip angle. The results of the linear method of Sedlacik et al. (left panel) show a larger spread compared to the results obtained with the RFT method.

FIG. 6 shows two-dimensional projections (e.g., SAG standing for sagittal, COR standing for coronal, and TRA standing for transversal) of a brain B₁ map acquired via MRI using an embodiment of the computing system of the present embodiments and comparing the brain B₁ map with the ground truth and a state-of-the-art fitting technique. The settings are the same detailed in FIG. 4. FIG. 6 shows contour plots of the B₁ magnetic field and the relative flip angle (e.g., relative FA) of one of the brain MRI, where the first row shows the results for the ground truth (e.g., obtained with AFI), the second row shows the results of the linear method of Sedlacik et al., and the third row shows the results of using a RFT regression algorithm with the computing system of the present embodiments.

FIG. 6 shows that the RFT regression algorithm is more accurate than the linear fitting, and, for example, the RFT regression algorithm may “fill in” voxels that are missing in the acquired data from satTFL. Further, the RFT regression algorithm is clearly superior when it comes to mapping the spatial inhomogeneity of the B₁ magnetic field, even for very small values of the flip angle (FA). This is a manifestation of the capacity of the RFT regression algorithm to leverage the geometric features introduced therein (e.g., the spatial correlation of the voxels) for a more accurate mapping of the B₁ magnetic field.

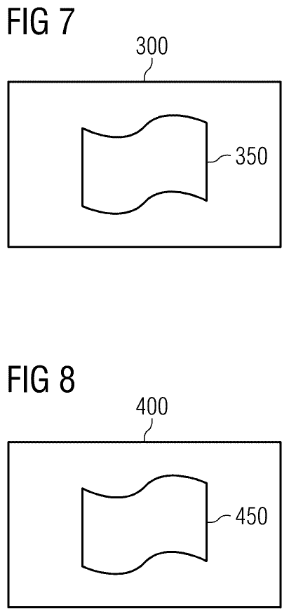

FIG. 7 shows a schematic block diagram illustrating a computer program product 300 according to an embodiment of the third aspect of the present embodiments. The computer program product 300 includes executable program code 350 configured to, when executed, perform the method according to any embodiment of the second aspect of the present embodiments (e.g., as has been described with respect to the preceding figures).

FIG. 8 shows a schematic block diagram illustrating a non-transitory computer-readable data storage medium 400 according to an embodiment of the fourth aspect of the present embodiments. The data storage medium 400 includes executable program code 450 configured to, when executed, perform the method according to any embodiment of the second aspect of the present embodiments (e.g., as has been described with respect to the preceding figures).

The non-transient computer-readable data storage medium may include, or consist of, any type of computer memory (e.g., semiconductor memory such as a solid-state memory). The data storage medium may also include, or consist of, a CD, a DVD, a Blu-Ray-Disc, an USB memory stick, or the like.

The previous description of the disclosed embodiments are merely examples of possible implementations, which are provided to enable any person skilled in the art to make or use the present invention. Various variations and modifications of these embodiments will be readily apparent to those skilled in the art, and the generic principles defined herein may be applied to other embodiments without departing from the spirit or scope of the present disclosure. Thus, the present invention is not intended to be limited to the embodiments shown herein, but the present invention is to be accorded the widest scope consistent with the principles and novel features disclosed herein. Therefore, the present invention is not to be limited except in accordance with the following claims.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A computing system for providing a mapping of a physical quantity on a biological tissue, the computing system comprising:
an input data interface configured to obtain data from the physical quantity on different spatial points of the biological tissue;
a computation module comprising:
a digitization unit configured to produce a digitized representation of the biological tissue in voxels, pixels, or voxels and pixels, wherein, based on the obtained data, a subset of the voxels, pixels, or voxels and pixels are assigned a value of the physical quantity;
a concatenation unit configured to spatially correlate the voxels, pixels, or voxels and pixels of the produced digitized representation of the biological tissue; and
a regression unit configured to:
process the information of the spatially correlated voxels, pixels, or voxels and pixels; and
generate a regression analysis of the physical quantity on the biological tissue; and
an output data interface configured to, based on the generated regression analysis, provide a mapping of the physical quantity on the biological tissue,
wherein a value of the physical quantity is assigned to each voxel, pixel, or voxel and pixel.

2. The computing system of claim 1, wherein the computation module further comprises a performance assessment unit that is configured to evaluate accuracy of the provided mapping based on a ground truth.

3. The computing system of claim 2, wherein the physical quantity is a radiofrequency magnetic field generated during magnetic resonance imaging (MRI).

4. The computing system of claim 3, wherein the MRI comprises ultrahigh field magnetic resonance imaging with parallel transmission imaging radiofrequency.

5. The computing system of claim 4, wherein the obtained data of the radiofrequency magnetic field is generated following a fast low angle shot magnetic resonance imaging.

6. The computing system of claim 3, wherein the ground truth comprises a mapping of the radiofrequency magnetic field acquired with actual flip angle imaging.

7. The computing system of claim 1, wherein the computation module further comprises a feature generating unit configured to compute a volume of the biological tissue, spatial coordinates of a center of mass of the biological tissue, or the volume and the spatial coordinates.

8. The computing system of claim 2, wherein the regression unit comprises an artificial intelligence entity configured to implement at least one machine learning regression algorithm.

9. The computing system of claim 8, wherein the at least one machine learning regression algorithm comprises a random forest tree algorithm, a gradient boosting algorithm, or the random forest tree algorithm and the gradient boosting algorithm.

10. The computing system of claim 8, wherein the machine learning regression algorithm uses as input a feature matrix comprising spatial components of each voxel, pixel, or voxel and pixel, and the value of the physical quantity assigned to the voxel, pixel, or voxel and pixel.

11. The computing system of claim 8, further comprising an optimization unit configured to use the evaluation of the performance assessment unit to optimize the machine learning regression algorithm.

12. The computing system of claim 1, wherein the biological tissue is brain tissue.

13. A medical scanning system comprising:
a magnetic resonance imaging scanner configured to perform magnetic resonance imaging scans; and
a computing system for providing a mapping of a physical quantity on a biological tissue, the computing system comprising:
an input data interface configured to obtain data from the physical quantity on different spatial points of the biological tissue;
a computation module comprising:
a digitization unit configured to produce a digitized representation of the biological tissue in voxels, pixels, or voxels and pixels, wherein, based on the obtained data, a subset of the voxels, pixels, or voxels and pixels are assigned a value of the physical quantity;
a concatenation unit configured to spatially correlate the voxels, pixels, or voxels and pixels of the produced digitized representation of the biological tissue; and
a regression unit configured to:
process the information of the spatially correlated voxels, pixels, or voxels and pixels; and
generate a regression analysis of the physical quantity on the biological tissue; and
an output data interface configured to, based on the generated regression analysis, provide a mapping of the physical quantity on the biological tissue,
wherein a value of the physical quantity is assigned to each voxel, pixel, or voxel and pixel,
wherein the physical quantity is a radiofrequency magnetic field generated during magnetic resonance imaging (MRI), and
wherein the computing system is configured to obtain data corresponding to the radio-frequency magnetic field from the magnetic resonance imaging scanner.

14. The medical scanning system of claim 13, wherein the magnetic resonance imaging scanner is further configured to perform ultrahigh field magnetic resonance imaging scans with parallel radio-frequency imaging.

15. A computer-implemented method for providing a mapping of a physical quantity on a biological tissue, the computer-implemented method comprising:
obtaining data from the physical quantity on different spatial points of the biological tissue;
producing a digitized representation of the biological tissue in voxels, pixels, or voxels and pixels, wherein a subset of the voxels, pixels, or voxels and pixels gets assigned a value of the physical quantity based on the obtained data;
spatially correlating the voxels, pixels, or voxels and pixels of the produced digitized representation of the biological tissue;
generating, based on the information of the spatially correlated voxels, pixels, or voxels and pixels, a regression analysis of the physical quantity on the biological tissue; and
providing, based on the generated regression analysis, a mapping of the physical quantity on the biological tissue,
wherein a value of the physical quantity is assigned to each voxel, pixel, or voxel and pixel.

16. The computer-implemented method of claim 15, further comprising:
creating a feature matrix comprising spatial components of each voxel, pixel, or voxel and pixel, and the value of the physical quantity assigned to voxel, pixel, or voxel and pixel,
wherein the feature matrix is used as input for generating the regression analysis.

17. A non-transient computer-readable storage medium that stores instructions executable by one or more processors to provide a mapping of a physical quantity on a biological tissue, the instructions comprising:
obtaining data from the physical quantity on different spatial points of the biological tissue;
producing a digitized representation of the biological tissue in voxels, pixels, or voxels and pixels, wherein a subset of the voxels, pixels, or voxels and pixels gets assigned a value of the physical quantity based on the obtained data;
spatially correlating the voxels, pixels, or voxels and pixels of the produced digitized representation of the biological tissue;
generating, based on the information of the spatially correlated voxels, pixels, or voxels and pixels, a regression analysis of the physical quantity on the biological tissue; and
providing, based on the generated regression analysis, a mapping of the physical quantity on the biological tissue,
wherein a value of the physical quantity is assigned to each voxel, pixel, or voxel and pixel.
